(19) United States
(12) Patent Application Publication
(10) Pub. No.: US 2024/0288407 A1
(43) Pub. Date: Aug. 29, 2024

(54) ANALYSIS OF NUCLEIC ACID MIXTURES

(71) Applicant: CUREVAC AG, Tübingen (DE)

(72) Inventors: Alexander SCHWENGER, Tübingen (DE); Stefan HEINZ, Tübingen (DE)

(73) Assignee: CureVac SE, Tübingen (DE)

(21) Appl. No.: 18/002,002

(22) PCT Filed: Jun. 15, 2020
(86) PCT No.: PCT/EP2020/066491

§ 371 Date: Dec. 15, 2022

(51) Int. Cl. G01N 30/86; C12Q 1/6806; G01N 30/02

(52) U.S. Cl. CPC G01N 30/8631; C12Q 1/6806; G01N 2030/027

(57) ABSTRACT

The present invention is concerned with a method for determining the integrity of a nucleic acid mixture comprising at least two nucleic acids with different sizes and the use of this method in quality control of a nucleic acid mixture.

This application is a national phase application under 35 U.S.C. § 371 of International Application No. PCT/EP2020/066491, filed Jun. 15, 2020, the entire contents of which are hereby incorporated by reference.

### FIELD OF THE INVENTION

The present invention relates to the field of nucleic acid analysis. More specifically, the present invention is inter alia concerned with a method for determining the integrity of a nucleic acid mixture comprising at least two nucleic acids with different sizes. The method involves subjecting the nucleic acid mixture comprising at least two nucleic acids with different sizes to a separation by size using a suitable method. After separation by size, the integrity of the nucleic acid with the largest size is determined, and the integrity of the nucleic acid mixture is then determined by assigning the integrity of the nucleic acid with the largest size to the integrity of the nucleic acid mixture. Furthermore, the present invention relates to the use of the method for determining the integrity of a nucleic acid mixture in quality control of a nucleic acid mixture.

### BACKGROUND OF THE INVENTION

The present invention is directed to a method for determining the integrity of a nucleic acid mixture comprising at least two nucleic acids with different sizes, in particular an RNA mixture, using inter alia a method of size separation, in particular electrophoresis or a chromatographic method, preferably HPLC. Further, the invention relates to the use of said method as a quality control of a nucleic acid mixture, in particular an RNA mixture.

Nucleic acid mixtures (used herein in the meaning of a nucleic acid mixture comprising at least two nucleic acids with different sizes, unless indicated otherwise) and in particular RNA mixtures may be used in therapy. Thus, RNA-based therapeutics can be used in immunotherapy, gene therapy, and genetic vaccination, amongst others. Such therapeutics can provide highly specific and individual treatment options for the therapy of a large variety of diseases.

For certain medical treatments and applications, it is desirable to apply a nucleic acid mixture, such as an RNA mixture, comprising different nucleic acids. Examples of such treatments based on an RNA mixture include the application of polyvalent RNA mixtures that provide protection against several serotypes of a pathogen, RNA mixtures that provide different antigens of a (single) pathogen, RNA mixtures that provide protection against several isoforms or variants of a (single) cancer antigen, RNA mixtures that provide different epitopes of a (single) antigen, RNA mixtures that contain a cancer specific and/or patient specific mixture of cancer antigens or epitopes thereof, RNA mixtures that encode different parts or units of an antibody, or any other therapeutically active RNA mixture (e.g., encoding different isoforms of a (single) enzyme for molecular therapy, different therapeutic proteins for treatment of an indication, where several proteins have to be supplemented).

Thus far, manufacturing of the RNA for RNA therapeutics mainly rests on the production of a single specific RNA at a time, such as e.g. an RNA encoding a single specific therapeutic target (see e.g. WO2016180430A1). For RNA mixtures, such individually produced RNAs are then typically mixed. The manufacturing process disclosed in WO2016180430A1, which is directed to the production of a single specific RNA at a time, has been approved by regulatory authorities and implements several quality controls (directed e.g. to the RNA presence, RNA integrity and RNA quantity) during or following the production of the RNA to eventually ensure a safe and effective RNA medicament. However, the different quality controls used for a single specific RNA cannot always be easily transferred to RNA mixtures.

One important quality attribute of an RNA produced for an RNA medicament is the integrity. RNA can degrade, which may be caused e.g. by heat, ribonucleases, pH or other factors, which reduces the integrity and, consequently, the functionality of the RNA. A typical method for determining the integrity of a single RNA is analytical HPLC. Analytical HPLC (e.g. RP-HPLC) is a technique, which allows the separation of complex samples through physicochemical interactions. This method is particularly well suited for the separation of large oligonucleotides such as RNA. HPLC provides a fast and reliable method of RNA analysis, especially in the detection and quantification of different RNAs. Thus, a typical parameter for a quality control is the integrity of the RNA, particularly RNA in a medicament, which may be determined immediately following the production of the RNA medicament, whereas another typical parameter for quality control can be the integrity of the RNA in the medicament after storage of the medicament for e.g. several months or even years. If the RNA has e.g. been degraded during storage over several years to an extent that would result in a functionality of the RNA that is too low, the medicament would not be suitable any more as medical after storage. This would be determined in a quality control directed to assessing the integrity after storage.

Analytical HPLC to determine the integrity of an RNA mixture is, however, not applicable using the methods disclosed in the prior art. For the analysis of an RNA mixture, it is required that the different RNAs of the RNA mixture and thus the mixture as such can be characterized in terms of integrity during or following production of the RNA mixture, and/or as quality control of a batch release. However, different RNAs in an RNA mixture usually elute at different time points, and may thus partially overlap, which strongly impedes the assessment of the quality/integrity of the RNA mixture via HPLC (illustrated in FIGS. 1A and B). For example, for the analysis of an RNA mixture comprising different RNAs, small degradation products of a larger RNA can elute within the main peak of a smaller, different RNA (FIG. 2). Accordingly, an assessment of the RNA integrity is neither possible for the individual RNA of the RNA mixture nor for the mixture as such.

Consequently, determination of integrity of RNA mixture-based therapeutics is strongly impeded with current separation methods (e.g. chromatography, electrophoresis) provided in the art. However, assays suitable for controlling quality and integrity in nucleic acid mixtures, in particular of RNA mixtures, are required in the field, particularly in the field of the production of medicaments based on nucleic acid mixtures, in particular RNA mixtures.

Therefore, it is an object of the present invention to provide a robust and reliable method for determining the integrity of a nucleic acid mixture and in particular of an RNA mixture, which can in particular be used in quality control of the nucleic acid mixture.

### SUMMARY OF THE INVENTION

The present invention solves the above need by inter alia providing a method for determining the integrity of a nucleic acid mixture including the separation of the nucleic acids in the mixture by size and determining the integrity of the nucleic acid with the largest size. It was found that the integrity of the nucleic acid with the largest size corresponds to the integrity of the nucleic acid mixture. Thus, the integrity of the nucleic acid mixture can be determined by assigning the integrity of the nucleic acid with the largest size to the nucleic acid mixture.

In the following, the aspects of the invention are described. Embodiments of these aspects are also disclosed.

### First Aspect: A Method for Determining the Integrity of a Nucleic Acid Mixture

In a first aspect, the present invention provides a method for determining the integrity of a nucleic acid mixture comprising at least two nucleic acids with different sizes, the method comprising the following steps:
a) subjecting the nucleic acid mixture comprising at least two nucleic acids with different sizes to a method for separation by size;
b) determining the integrity of the nucleic acid with the largest size; and
c) determining the integrity of the nucleic acid mixture by assigning the integrity of the nucleic acid with the largest size determined in step b) to the nucleic acid mixture.

In principle, any analytical method which separates nucleic acids by size is suitable. Analytical methods which separate a mixture of different compounds, in particular nucleic acids, depending on their sizes are known in the art. In an embodiment of the first aspect, the method for separation by size is a chromatographic method or electrophoresis.

In a preferred embodiment of the first aspect, the chromatographic method is liquid chromatography.

The liquid chromatography may be selected from the group consisting of normal phase liquid chromatography, reversed phase liquid chromatography (RPLC), ion-exchange liquid chromatography, ion-pair reversed-phase liquid chromatography (IP-RPLC), size-exclusion chromatography (SEC), high-performance liquid chromatography (HPLC), reversed phase high-performance liquid chromatography (RP-HPLC), ion-pair reversed-phase high-performance liquid chromatography (IP-RP-HPLC), ultra-high performance liquid chromatography (UHPLC), hydrophilic interaction chromatography (HIC), hydrophilic interaction liquid chromatography (HILIC), and combinations thereof. In a preferred embodiment, the liquid chromatography is high-performance liquid chromatography (HPLC), reversed phase high-performance liquid chromatography (RP-HPLC) or ion-pair reversed-phase high-performance liquid chromatography (IP-RP-HPLC). In an even more preferred embodiment, the liquid chromatography is reversed phase high-performance liquid chromatography (RP-HPLC) or ion-pair reversed-phase high-performance liquid chromatography (IP-RP-HPLC). In the most preferred embodiment, the liquid chromatography is reversed phase high-performance liquid chromatography (RP-HPLC).

The stationary phase of the liquid chromatography may be a monolithic stationary phase or a particulate stationary phase. The monolithic or particulate stationary phase can be silica-based or organic polymer-based. In a preferred embodiment, the monolithic or particulate stationary phase is organic polymer-based.

In a more preferred embodiment, the monolithic stationary phase or the particulate stationary phase comprises a poly(styrene-divinylbenzene) matrix. The poly(styrene-divinylbenzene) matrix can be derivatized with functional groups. In an even more preferred embodiment, the stationary phase is a poly(styrene-divinylbenzene) monolithic stationary phase. The poly(styrene-divinylbenzene) monolithic stationary phase can be derivatized with functional groups.

The nucleic acid mixture of the method of the first aspect comprises at least two nucleic acids with different sizes.

In an embodiment thereof, the sizes of the at least two nucleic acids of the nucleic acid mixture differ by at least 5, 10, 15, 20, 25, 30, 40, 50, 60, 70, 80, 90, 100, 125, 150, 175 or 200 nucleotides. In a specific embodiment, the sizes of the at least two nucleic acids of the nucleic acid mixture differ by at least 150 nucleotides. In another specific embodiment, the sizes of the at least two nucleic acids of the nucleic acid mixture differ by at least 100 nucleotides. In another embodiment, the sizes of the at least two nucleic acids of the nucleic acid mixture differ by at least 50 nucleotides. In a particularly preferred embodiment, the sizes of the at least two nucleic acids of the nucleic acid mixture differ by at least 30 nucleotides. In an even more preferred embodiment, the sizes of the at least two nucleic acids of the nucleic acid mixture differ by at least 20 nucleotides.

In another embodiment thereof, the at least two nucleic acids in the nucleic acid mixture differ in their sizes due to differences in their molecular weights of at least about 10 g/mol. This difference in the molecular weights may be due to a difference in the size, in particular the difference by at least 20 nucleotides (the most preferred embodiment) as mentioned above, but it may also be due to a difference in the types of nucleotides present in the different nucleic acids.

In an embodiment of the first aspect, the integrity of the nucleic acid with the largest size is determined by comparing the amount of the nucleic acid with the largest size determined prior to the separation by size (e.g. determined immediately after production of the nucleic acid mixture or immediately after the production of the nucleic acid with the largest size prior to mixing it with other nucleic acids to obtain the nucleic acid mixture) to the actual amount of the nucleic acid with the largest size determined after the separation by size.

Thus, the integrity of the nucleic acid with the largest size may be determined by comparing the amount of the nucleic acid with the largest size at two different time points t₀ and t₁, namely a time point before separation (t₀) and a time point after separation (t₁). The time point before separation to is preferably during production, after production or as part of a batch release control of the nucleic acid with the largest size and/or the nucleic acid mixture comprising at least two nucleic acids with different sizes including the nucleic acid with the largest size. The time period between the two time points t₀ and t₁, i.e. before and after separation, can be hours, days, months or years. In other words, it is an integrity criterion when producing the nucleic acid mixture that the amounts of the different nucleic acids comprised therein are known, and this determination of the amounts is usually an integral part of quality control during production. This of course particularly applies if the production of the nucleic acid mixture is completed, e.g. in the form of a final quality control step of the produced nucleic acid mixture. Amongst other parameters, the amounts of the different nucleic acids in the produced nucleic acid mixture are recorded and are thus known prior to e.g. (i) storing the nucleic acid mixture, (ii) shipping the nucleic acid mixture and/or (iii) complexing the nucleic acid mixture e.g. with a lipid nanoparticle. This known amount of the nucleic acid with the largest size is the amount at time point to. It is typically saved as a parameter of the nucleic acid mixture (e.g. via a barcode at the nucleic acid mixture linked to a database saving the parameters). After e.g. (i) storage, (ii) shipping and/or (iii) complexing, it is of course of interest whether the integrity of the nucleic acid mixture is still satisfactory, in particular whether it still complies with regulatory requirements. For this reason, the amount (referred to as the “actual amount” herein) of the nucleic acid with the largest size is again determined at t₁ (i.e. after e.g. (i) storage, (ii) shipping and/or (iii) complexing). If the nucleic acid mixture is an RNA mixture, the RNA comprised in this mixture is typically produced via RNA in vitro transcription.

Accordingly, in an embodiment, the amount of the nucleic acid with the largest size determined prior to the separation by size is known from the production of the nucleic acid with the largest size and/or the production of the nucleic acid mixture of the at least two nucleic acids with different sizes. It goes without saying that the sample comprising the nucleic acid with the largest size, for which the integrity was determined at t₀ and for which the amount is known prior to the separation by size, is also the sample (or a part of that sample is used) that is subjected to the separation by size in order to obtain the actual amount, i.e. the amount at t₁.

In an embodiment, the actual amount of the nucleic acid with the largest size is determined by analysing the fraction of the nucleic acid with the largest size obtained by a chromatographic method or electrophoretic method. In a preferred embodiment, the analysing comprises determining the area under the peak of the fraction of the nucleic acid with the largest size obtained by a chromatographic method. In another embodiment, the area under the peak of the fraction of the nucleic acid with the largest size is integrated. The integrated peak of the fraction of the nucleic acid with the largest size is then typically indicative of the actual amount of the nucleic acid with the largest size. The amount can be determined by measuring a reference standard or any other suitable quantitative method known in the art. The integrity may be indicated in % integrity.

In an embodiment, the nucleic acid mixture comprising at least two nucleic acids with different sizes is comprised in a formulation comprising at least one further component, wherein this formulation may e.g. be a pharmaceutical composition for administration to an animal or a human subject. In an embodiment, the at least one further component is selected from the group consisting of a lipid, a protein, a peptide, a cationic compound, a polycationic compound, and combinations thereof. In a preferred embodiment, the at least one further component is a lipid nanoparticle or a cationic or polycationic peptide or a cationic or polycationic protein. In a particularly preferred embodiment, the at least one further component is a lipid nanoparticle. In this particularly preferred embodiment, one may also refer to the nucleic acid mixture being a lipid nanoparticle-encapsulated nucleic acid mixture.

When the nucleic acid mixture is comprised in a formulation comprising at least one further component, the nucleic acid mixture will typically be separated from the at least one further component prior to step a), i.e. prior to subjecting the nucleic acid mixture comprising at least two nucleic acids with different sizes to a method for separation by size. Suitable methods for separating the nucleic acid mixture from the at least one further component in the formulation are known in the art.

Such methods can typically include the contacting of the formulation comprising the nucleic acid mixture and the at least one further component with salt in the form of a “high salt treatment step” to release the nucleic acids. This is a preferred method for formulations based on polymers/peptides. A suitable salt for performing this “high salt treatment step” may be NaCl. The salt, preferably NaCl, may be used in a range from 500 mM to 5 M, preferably from 1 M to 5 M, more preferably from 0.75 M to 3 M, most preferably NaCl is used in a concentration of about 1 to 2 M. If protamine is present, 1.5 M NaCl is a suitable concentration. Alternatively, such methods can typically include the contacting of the formulation comprising the nucleic acid mixture and the at least one further component with a detergent in the form of a “detergent treatment step” to release the nucleic acids. This is a preferred method for formulations based on lipid nanoparticle (LNP)-encapsulated nucleic acid mixtures. A suitable detergent for performing this “detergent treatment step” may be Triton-X or Tween. The detergent may be used in a range from 500 mM to 5 M, preferably from 1 M to 5 M.

The step of dissociating the complexed RNA may additionally require the incubation at elevated temperatures. A suitable temperature for the dissociation step may be at about 60° C. to 95° C., preferably at about 70° C. to 90° C., more preferably at about 80° C. to 90° C., most preferably at about 85° C.

In an embodiment, the nucleic acid is DNA or RNA. In a preferred embodiment, the nucleic acid is RNA. In a particularly preferred embodiment, the RNA is mRNA.

In a preferred embodiment of the first aspect, there is provided a method for determining the integrity of a nucleic acid mixture comprising at least two nucleic acids with different sizes, the method comprising the following steps:
a) subjecting the nucleic acid mixture comprising at least two nucleic acids with different sizes to a method for separation by size, wherein the method for separation by size is a chromatographic method, preferably RP-HPLC;
b) determining the integrity of the nucleic acid with the largest size; and
c) determining the integrity of the nucleic acid mixture by assigning the integrity of the nucleic acid with the largest size determined in step b) to the nucleic acid mixture.

In another preferred embodiment of the first aspect, there is provided a method for determining the integrity of a nucleic acid mixture comprising at least two nucleic acids with different sizes, the method comprising the following steps:
a) subjecting the nucleic acid mixture comprising at least two nucleic acids with different sizes to a method for separation by size, wherein the method for separation by size is a chromatographic method, preferably RP-HPLC;
b) determining the integrity of the nucleic acid with the largest size; and
c) determining the integrity of the nucleic acid mixture by assigning the integrity of the nucleic acid with the largest size determined in step b) to the nucleic acid mixture;
wherein the nucleic acid is RNA, preferably mRNA.

In another preferred embodiment of the first aspect, there is provided a method for determining the integrity of a nucleic acid mixture comprising at least two nucleic acids with different sizes, the method comprising the following steps:
a) subjecting the nucleic acid mixture comprising at least two nucleic acids with different sizes to a method for separation by size, wherein the method for separation by size is a chromatographic method, preferably RP-HPLC;
b) determining the integrity of the nucleic acid with the largest size by comparing the amount of the nucleic acid with the largest size determined prior to the separation by size to the actual amount of the nucleic acid with the largest size determined after the separation by size; and
c) determining the integrity of the nucleic acid mixture by assigning the integrity of the nucleic acid with the largest size determined in step b) to the nucleic acid mixture;
wherein the nucleic acid is RNA, preferably mRNA.

In another preferred embodiment of the first aspect, there is provided a method for determining the integrity of a nucleic acid mixture comprising at least two nucleic acids with different sizes, the method comprising the following steps:
a) subjecting the nucleic acid mixture comprising at least two nucleic acids with different sizes to a method for separation by size, wherein the method for separation by size is a chromatographic method, preferably RP-HPLC;
b) determining the integrity of the nucleic acid with the largest size; and
c) determining the integrity of the nucleic acid mixture by assigning the integrity of the nucleic acid with the largest size determined in step b) to the nucleic acid mixture;
wherein the nucleic acid mixture is comprised in a formulation comprising at least one further component, wherein the at least one further component is preferably a lipid nanoparticle, and wherein the nucleic acid mixture is separated from the at least one further component prior to step a); and
wherein the nucleic acid is RNA, preferably mRNA.

### Second Aspect: Use of the Method According to the First Aspect of the Present Invention

In a second aspect, the present invention relates to the use of the method according to the first aspect (including all embodiments as described above) in a quality control of a nucleic acid mixture.

For nucleic acids in general including a nucleic acid mixture, it is an important quality control to determine the integrity of the nucleic acids (e.g. between production and administration, between production and shipping, between production and packaging or after production and before administration). Thus, in an embodiment, the quality control of the second aspect is the control of the integrity of a nucleic acid mixture.

In an embodiment, the nucleic acid mixture comprising at least two nucleic acids with different sizes is comprised in a formulation comprising at least one further component. The at least one further component may be selected from the group consisting of a lipid, a protein, a peptide, a cationic compound, a polycationic compound, and combinations thereof. In a preferred embodiment, the at least one further component is a lipid nanoparticle or a cationic or polycationic peptide or a cationic or polycationic protein. In a particularly preferred embodiment, the at least one further component is a lipid nanoparticle. In this particularly preferred embodiment, one may also refer to the nucleic acid mixture being a lipid nanoparticle-encapsulated nucleic acid mixture.

In an embodiment, the above formulation is a pharmaceutical formulation, which is for use in therapy and/or prevention. In an embodiment, the therapy relates to an immunotherapy or a gene-therapy. In an embodiment, the prevention relates to a prevention of a disease by vaccination. Thus, the pharmaceutical formulation may be for use in immunotherapy, gene therapy and/or vaccination.

In an embodiment, the nucleic acid is DNA or RNA. In a preferred embodiment, the nucleic acid is RNA. In a particularly preferred embodiment, the RNA is mRNA.

### BRIEF DESCRIPTION OF THE DRAWINGS

The figures shown in the following are merely illustrative and shall describe the present invention in a further way.

These figures shall not be construed to limit the present invention thereto.

FIG. 1A, B Representative chromatogram of a mixture of three different RNAs 1, 2 and 3 eluting at different time points according to their different sizes. The peaks of the different RNAs partially overlap.

FIG. 2 Representative chromatogram of a mixture of two different RNAs 1 and 2: Small degradation products of the larger RNA 2 co-elute within the main peak of RNA 1 as indicated by the box 3.

FIG. 3 Representative chromatogram showing the determination of the relative peak area for determining the amount of integer RNA.

FIG. 4 Integrity of RNAs R1-R5 differing in their sizes after heating to 85° C. in order to promote RNA degradation as measured by RP-HPLC based on relative peak area. RNA size and RNA integrity (in %) show a linear correlation.

FIG. 5A HPLC chromatograms showing the course of degradation of the RNA mixture at t=1 min.

FIG. 5B HPLC chromatograms showing the course of degradation of the RNA mixture at t=10 min.

FIG. 5C HPLC chromatograms showing the course of degradation of the RNA mixture at t=40 min.

FIG. 6 Integrity of RNA species R1 (“first peak”) and R2 (“last peak”) plotted over time. The integrity of the last peak RNA follows the course of degradation, while the integrity of the first peak RNA does not follow the course of degradation.

FIG. 7 shows the peak area (normalized over all three samples) of RNA R2 and RNA R1, determined either in individual samples (R1, R2) or in the mixture of R1 and R2 (R1-mix, R2-mix). The normalized peak areas for the RNA R2, the RNA with the larger size, show an almost identical course of degradation when determined in the RNA mixture (comprising R1 and R2, see R2-mix) as well as when measured in the individual RNA R2 sample (R2). In contrast, the normalized peak area for the RNA R1 determined for the individual RNA R1 sample (R1) shows an expected course of degradation, while the values obtained from the RNA mixture (comprising R1 and R2, see R1-mix), evaluated based on the normalized RNA R1 peak area, strongly deviate from the expected values.

FIG. 8 Degradation levels of the five different RNAs depending on the ratio mix.

FIG. 9 Chromatograms of the different ratio mixtures of the five different RNAs. The last peak in each chromatogram corresponds to RNA R5.

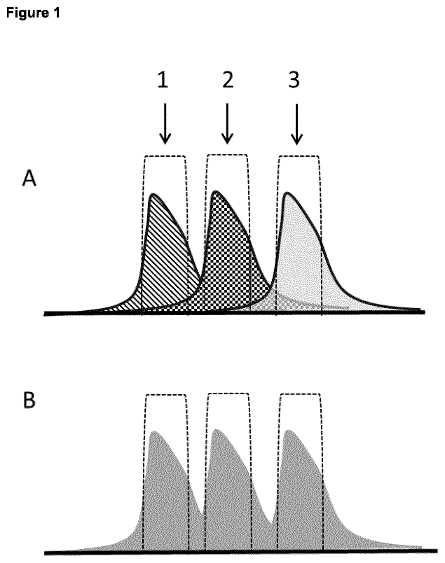

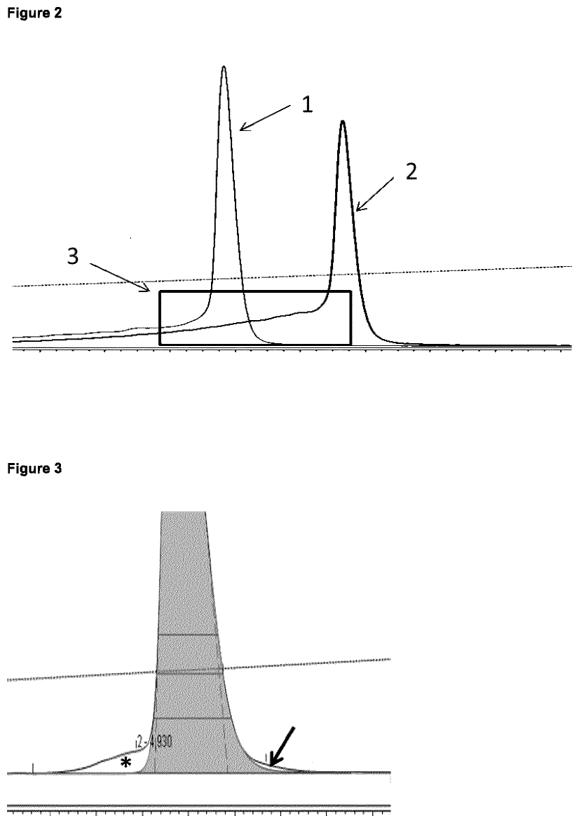

### DEFINITIONS

For the sake of clarity and readability the following definitions are provided. Any technical feature mentioned for these definitions may be read on each and every embodiment of the invention. Additional definitions and explanations may be specifically provided in the context of these embodiments.

As used in the specification and the claims, the singular forms of “a” and “an” also include the corresponding plurals unless the context clearly dictates otherwise.

The term “about” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the present invention, the term “consisting of” is considered to be a preferred embodiment of the term “comprising”. If hereinafter a group is defined to comprise at least a certain number of embodiments, this is also meant to encompass a group which preferably consists of these embodiments only.

The term “nucleic acid” means any DNA- or RNA-molecule and is used synonymous with polynucleotide. A “nucleic acid” as used herein may comprise a “modified nucleotide” as defined herein.

The “nucleic acid with the largest size” in a nucleic acid mixture comprising at least two nucleic acids with different sizes is the nucleic acid with the largest molecular weight. Put in other words, the nucleic acid with the largest size is usually the nucleic acid with the largest number of basepairs or bases. However, in view of the afore-mentioned definition, a difference in the size may also be the result of a difference in the type of nucleotides present in the at least two given nucleic acids. In view of the different nucleotides having different molecular weights, differences in the types of nucleotides may also result in a difference in the molecular weight, ultimately again giving rise to a nucleic acid with the largest size in a nucleic acid mixture.

The term “modified nucleotides” as used herein will be recognized and understood by the person of ordinary skill in the art, and is for example intended to comprise nucleotides that comprise a modification. For example, any nucleotide different from G, C, U, T, A may be regarded as “modified nucleotide”. Such modified nucleotides may be incorporated during RNA in vitro transcription of the RNA (e.g. by using pseudouridine (ψ), N1-methylpseudouridine (m1ψ), or 5-methylcytosine, and 5-methoxyuridine instead of uracil in the nucleotide mixture of the transcription reaction). Modified nucleotides known in the art comprise 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deazaadenosine, pseudouridine, N1-methylpseudouridine, N1-ethylpseudouridine, 2-thiouridine, 4′-thiouridine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 2′-0-methyl uridine, pseudouridine (ψ), N1-methylpseudouridine (m1ψ), 5-methylcytosine, and 5-methoxyuridine.

The term “nucleic acid mixture” as used herein refers to a plurality of different nucleic acid molecules comprised in one mixture or composition. A nucleic acid mixture contains at least two different nucleic acids. The term does not encompass the presence of several identical nucleic acid molecules. In other words, a plurality of identical nucleic acid molecules is not a nucleic acid mixture as used herein but a single nucleic acid (composition). In the context of the present invention, a nucleic acid mixture contains at least two nucleic acids with different sizes.

The term “DNA” is the usual abbreviation for deoxyribonucleic acid. It is a nucleic acid molecule, i.e. a polymer consisting of nucleotide monomers. These nucleotides are usually deoxy-adenosine-monophosphate, deoxy-thymidine-monophosphate, deoxy-guanosine-monophosphate and deoxy-cytidine-monophosphate monomers or analogs thereof which are—by themselves—composed of a sugar moiety (deoxyribose), a base moiety and a phosphate moiety, and polymerize by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA-sequence. DNA may be single stranded or double stranded. In the double stranded form, the nucleotides of the first strand typically hybridize with the nucleotides of the second strand, e.g. by A/T-base-pairing and G/C-base-pairing.

The term “DNA mixture” as used herein refers to a plurality of different DNAs comprised in one mixture or composition. A DNA mixture contains at least two different DNAs. The term does not encompass the presence of several identical DNAs. In other words, a plurality of identical DNAs is not a DNA mixture as used herein but a single DNA (composition). In the context of the present invention, a DNA mixture contains at least two DNAs with different sizes.

The term “RNA” is the usual abbreviation for ribonucleic acid. It is a nucleic acid molecule, i.e. a polymer consisting of nucleotide monomers. These nucleotides are usually adenosine-monophosphate, uridine-monophosphate, guanosine-monophosphate and cytidine-monophosphate monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA-sequence. The term “RNA” may refer to a molecule or to a molecule species selected from the group consisting of long-chain RNA, coding RNA, non-coding RNA, single stranded RNA (ssRNA), double stranded RNA (dsRNA), linear RNA (linRNA), circular RNA (circRNA), messenger RNA (mRNA), RNA oligonucleotides, small interfering RNA (siRNA), small hairpin RNA (shRNA), antisense RNA (asRNA), CRISPR/Cas9 guide RNAs, riboswitches, immunostimulating RNA (isRNA), ribozymes, aptamers, ribosomal RNA (rRNA), transfer RNA (tRNA), viral RNA (vRNA), retroviral RNA or replicon RNA, small nuclear RNA (snRNA), small nucleolar RNA (snoRNA), microRNA (miRNA), circular RNA (circRNA), and a Piwi-interacting RNA (piRNA). Preferred in the context of the invention is any type of therapeutic RNA. “Therapeutic RNA” is to be understood as relating to RNA that is suitable for use in the human or animal body for a medical purpose, i.e. it has a clinical grade, particularly when it comes to parameters such as purity, integrity, as well as concerning the underlying production methods that must comply with (c)GMP conditions. Therapeutic RNA can be used in immunotherapy, gene therapy and (genetic) vaccination.

The term “messenger RNA” (mRNA) refer to one type of RNA molecule. In vivo, transcription of DNA usually results in the so-called premature RNA which has to be processed into so-called messenger RNA, usually abbreviated as mRNA. Processing of the premature RNA, e.g. in eukaryotic organisms, comprises a variety of different posttranscriptional modifications such as splicing, 5′-capping, polyadenylation, export from the nucleus or the mitochondria and the like. The sum of these processes is also called maturation of mRNA. The mature messenger RNA usually provides the nucleotide sequence that may be translated into an amino acid sequence of a particular peptide or protein. Typically, a mature mRNA comprises a 5′ cap, a 5′UTR, an open reading frame, a 3′UTR and a poly(A) or a poly(C) sequence. In the context of the present invention, an mRNA may also be an artificial molecule, i.e. a molecule not occurring in nature. This means that the mRNA in the context of the present invention may, e.g., comprise a combination of a 5′UTR, open reading frame, 3′UTR and poly(A) sequence, which does not occur in this combination in nature.

The term “RNA mixture” as used herein refers to a plurality of different RNAs comprised in one mixture or composition. An RNA mixture contains at least two different RNAs. The term does not encompass the presence of several identical RNAs. In other words, a plurality of identical RNAs is not an RNA mixture as used herein but a single RNA (composition). In the context of the present invention, an RNA mixture contains at least two RNAs with different sizes.

The term “integrity” generally describes whether the complete nucleic acid sequence is present in a nucleic acid that is present in a sample. Low integrity could be due to, amongst others, degradation, cleavage, incorrect or incomplete chemical synthesis, incorrect base pairing, integration of modified nucleotides or the modification of already integrated nucleotides, lack of or incomplete capping, lack of or incomplete polyadenylation, or incomplete transcription.

The term “RNA in vitro transcription” relates to a process wherein RNA is synthesized from a DNA template in a cell-free system (in vitro). DNA, preferably a linear DNA (e.g. linearized plasmid DNA, linearized dbDNA), is used as a template for the generation of RNA transcripts. A DNA template for RNA in vitro transcription may be obtained by cloning of a nucleic acid, in particular cDNA corresponding to the respective RNA to be in vitro transcribed, and introducing it into an appropriate vector for RNA in vitro transcription, e.g. into plasmid DNA.

The term “RNA polymerase” refers to any enzyme which catalyzes the transcription of a DNA template into RNA. A “DNA-dependent RNA polymerase” can only catalyze the transcription of RNA from a DNA template.

The term “buffer” denotes a weak acid or base used to maintain acidity (pH) of a solution near a chosen value after the addition of another acid or base. Hence, the function of a buffer substance is to prevent rapid change in pH when acids or bases are added to the solution.

Nucleic acids may be present in complexed form with e.g. a lipid, a protein, a peptide, a cationic compound, a polycationic compound, and combinations thereof. For example, the nucleic acids according to the invention may be all or partially complexed with one or more cationic or polycationic compounds, preferably with cationic or polycationic polymers, cationic or polycationic peptides or proteins, e.g. protamine, cationic or polycationic polysaccharides and/or cationic or polycationic lipids. Also, the nucleic acids according to the invention can all or partially be complexed with lipids to form one or more liposomes, lipoplexes, or lipid nanoparticles. Therefore, the mixture of nucleic acids can comprise liposomes, lipoplexes, and/or lipid nanoparticles. In the context of the present invention, the nucleic acid, particularly the RNA, is preferably encapsulated in or complexed with lipid nanoparticles, as described in the following. Whenever reference is made in the following to a nucleic acid or RNA, this is to be understood for the method of the present invention as referring to a nucleic acid mixture or an RNA mixture, respectively.

Lipid-based formulations are used for delivery of therapeutic nucleic acids due to their biocompatibility and their ease of large-scale production. Cationic lipids have been widely studied as synthetic materials for delivery of nucleic acids, e.g. RNA, to form liposomes, lipid nanoparticles (LNPs), lipoplexes, and/or nanoliposomes.

The liposomes, lipid nanoparticles (LNPs), lipoplexes, and/or nanoliposomes—incorporated nucleic acid (e.g. RNA) may be completely or partially located in the interior space of the liposomes, lipid nanoparticles (LNPs), lipoplexes, and/or nanoliposomes, within the lipid layer/membrane, or associated with the exterior surface of the lipid layer/membrane. The incorporation of a nucleic acid into liposomes/LNPs is also referred to herein as “encapsulation” wherein the nucleic acid, e.g. the RNA, is entirely contained within the interior space of the liposomes, lipid nanoparticles (LNPs), lipoplexes, and/or nanoliposomes. The purpose of incorporating nucleic acid into liposomes, lipid nanoparticles (LNPs), lipoplexes, and/or nanoliposomes is to protect the nucleic acid, preferably RNA, from an environment which may contain enzymes or chemicals or conditions that degrade nucleic acid and/or systems or receptors that cause the rapid excretion of the nucleic acid. Moreover, incorporating nucleic acid, preferably RNA, into liposomes, lipid nanoparticles (LNPs), lipoplexes, and/or nanoliposomes may promote the uptake of the nucleic acid, and hence, may enhance the therapeutic effect of the nucleic acid.

In this context, the terms “complexed” or “associated” refer to the essentially stable combination of nucleic acid with one or more lipids into larger complexes or assemblies without covalent binding.

The term “lipid nanoparticle”, also referred to as “LNP”, is not restricted to any particular morphology, and include any morphology generated when a cationic lipid and optionally one or more further lipids are combined, e.g. in an aqueous environment and/or in the presence of a nucleic acid, e.g. an RNA. For example, a liposome, a lipid complex, a lipoplex and the like are within the scope of a lipid nanoparticle (LNP).

Liposomes, lipid nanoparticles (LNPs), lipoplexes, and/or nanoliposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter.

LNPs of the invention are suitably characterized as microscopic vesicles having an interior aqua space sequestered from an outer medium by a membrane of one or more bilayers. Bilayer membranes of LNPs are typically formed by amphiphilic molecules, such as lipids of synthetic or natural origin that comprise spatially separated hydrophilic and hydrophobic domains. Bilayer membranes of the liposomes can also be formed by amphophilic polymers and surfactants (e.g., polymerosomes, niosomes, etc.). In the context of the present invention, an LNP typically serves to transport the nucleic acid, preferably the RNA, to a target tissue.

A typical liposome or lipid nanoparticles (LNPs) comprise: (a) the nucleic acid, preferably the RNA (b) at least one cationic or ionizable lipid, (c) an aggregation reducing agent (such as polyethylene glycol (PEG) lipid or PEG-modified lipid), (d) optionally, a non-cationic lipid (such as a neutral lipid), and (e) optionally, a sterol.

Therefore, the nucleic acids of the method according to the present invention can be complexed with cationic lipids and/or neutral lipids and thereby form liposomes, lipid nanoparticles, lipoplexes or neutral lipid-based nanoliposomes. Preferably, the nucleic acids of the method according to the present invention are complexed with lipid nanoparticles or cationic or polycationic peptides or cationic or polycationic protein.

“Electrophoresis” is a general term that describes the migration and separation of charged particles (ions) under the influence of an electric field. An electrophoretic system consists of two electrodes of opposite charge (anode, cathode), connected by a conducting medium called an electrolyte. Electrophoresis is used in laboratories to separate macromolecules based on size. In electrophoresis of nucleic acids, a negative charge is applied so that nucleic acids such as DNA and RNA migrate to different extents depending on their size.

“Chromatography” is a technique in analytic chemistry used to separate the components in a mixture, to identify each component, and to quantify each component, such as a nucleic acid or a mixture of nucleic acids. The mixture is dissolved in a fluid called the mobile phase, which carries it through a structure holding another material called the stationary phase. The various constituents of the mixture travel at different speeds, causing them to separate. The separation is based on differential partitioning between the mobile and stationary phases. Subtle differences in a compound's partition coefficient result in differential retention on the stationary phase and thus affect the separation. Large molecules show a higher retention on the stationary phase than smaller molecules, resulting in a separation by size. In the resulting chromatogram, the larger molecules will thus show higher retention times than the smaller molecules.

“Normal phase liquid chromatography” is a chromatography type that uses a polar stationary phase and a nonpolar mobile phase for the separation of usually rather polar compounds.

“Reverse phase liquid chromatography” is a chromatography type that uses a non-polar (or hydrophobic) stationary phase and a polar mobile phase for the separation of usually rather non-polar compounds. Retention can be adjusted by altering the aqueous-to-organic content of the mobile phase. Reversed phase HPLC works with hydrophobic interactions which result from repulsive forces between a relatively polar solvent, the relatively non-polar analyte, and the non-polar stationary phase (reversed phase principle). The retention time on the column is therefore longer for molecules which are more non-polar in nature, allowing polar molecules to elute more readily. The retention time is increased by the addition of polar solvent to the mobile phase and decreased by the addition of more hydrophobic solvent. As larger molecules show more interaction with the hydrophobic stationary phase, their retention times will be longer as compared to smaller molecules.

“Ion-exchange chromatography” (or ion chromatography) is a chromatography process that separates ions and polar molecules based on their affinity to the ion exchanger. In this type of chromatography, the stationary phase is positively charged and negatively charged molecules are loaded to be attracted to it (anion exchange chromatography) or vice versa (cation exchange chromatography). In case of negatively charged nucleic acids, a positively charged stationary phase is usually used, i.e. anion exchange chromatography. As larger molecules show more ionic interaction with stationary phase, their retention times will be longer as compared to smaller molecules.

“Ion-pair reversed phase liquid chromatography” relies upon the addition of a counter ion to the mobile phase in order to promote the formation of ion-pairs with charged analytes. These counter ion reagents are usually ionic compounds that contain an alkyl chain that imparts certain hydrophobicity so that the ion-pair can be retained on a reversed-phase column. When used with common hydrophobic HPLC phases in the reverse phase mode, they can be used to selectively increase the retention of charged analytes. A negatively charged reagent having a charge opposite to the analyte of interest, such as one of the alkyl sulphonic acids can be used to retain positively charged ionic bases. Similarly, a positively charged reagent, such as tetrabutyl ammonium chloride, can be used to retain negatively charged ionic acids.

“Hydrophilic interaction chromatography (HIC)” or “hydrophilic interaction liquid chromatography (HILIC)”, is a variant of normal phase liquid chromatography that partly overlaps with other chromatographic applications such as ion chromatography and reversed phase liquid chromatography. HILIC uses hydrophilic stationary phases (for example, silica or a polar bonded phase) with reversed-phase type eluents. Most commonly, separations are carried out using 5-40% water (or aqueous buffers); the technique also is compatible with gradient elution. In HILIC, hydrophilic, polar, and charged compounds are retained preferentially compared with hydrophobic neutral compounds—the opposite of reversed-phase LC. Increasing the proportion of the aqueous component of the mobile phase in HILIC generally gives reduced retention. The order of solute elution resembles that of normal-phase chromatography and indeed, HILIC sometimes is called “aqueous normal-phase chromatography”; some studies suggest there can be at least some similarity in the separation mechanism of the two techniques. The surface of commonly used silica columns in HILIC is deactivated by the presence of the significant amounts of water in the mobile phase in comparison with regular normal-phase chromatography. As a result, fewer peak shape problems are obtained for strongly polar solutes in comparison to normal-phase separations.

“Size exclusion chromatography” (SEC, also known as gel filtration chromatography) separates molecules based on their size by filtration through a gel. The gel consists of spherical beads containing pores of a specific size distribution. Separation occurs when molecules of different sizes are included or excluded from the pores within the matrix. Small molecules diffuse into the pores and their flow through the column is retarded according to their size, while large molecules do not enter the pores and are eluted in the column's void volume. Consequently, molecules separate based on their size as they pass through the column and are eluted in order of decreasing molecular weight (MW).

“High-performance liquid chromatography” (HPLC; formerly referred to as high-pressure liquid chromatography) relies on (high pressure) pumps to pass a pressurized liquid solvent containing the sample mixture through a column filled with a solid adsorbent material. Each component in the sample interacts slightly differently with the adsorbent material, causing different flow rates for the different components and leading to the separation of the components as they flow out the column. HPLC is distinguished from traditional (“low pressure”) liquid chromatography because operational pressures are significantly higher (50-350 bar), while ordinary liquid chromatography typically relies on the force of gravity to pass the mobile phase through the column. Due to the small sample amount separated in analytical HPLC, typical column dimensions are 2.1-4.6 mm diameter, and 30-250 mm length. Also HPLC columns are made with smaller sorbent particles (2-50 micrometer in average particle size). This gives HPLC superior resolving power when separating mixtures, which is why it is a popular chromatographic technique. The schematic of an HPLC instrument typically includes a sampler, pumps, and a detector. The sampler brings the sample mixture into the mobile phase stream which carries it into the column. The pumps deliver the desired flow and composition of the mobile phase through the column. The detector generates a signal proportional to the amount of sample component emerging from the column, hence allowing for quantitative analysis of the sample components. A digital microprocessor and user software control the HPLC instrument and provide data analysis. Some models of mechanical pumps in a HPLC instrument can mix multiple solvents together in ratios changing in time, generating a composition gradient in the mobile phase. Various detectors are in common use, such as UV/Vis, photodiode array (PDA) or based on mass spectrometry. Most HPLC instruments also have a column oven that allows for adjusting the temperature the separation is performed at.

The above mentioned different types of liquid chromatography, i.e. normal phase, reversed phase, ion-exchange, ion-pair reversed phase, hydrophilic interaction and size exclusion liquid chromatography can be conducted as high-performance liquid chromatography as well.

“Ultra-high-performance liquid chromatography” (UHPLC) is a further development of HPLC. As compared to HPLC, UHPLC is characterized by smaller size columns (usually with an internal diameter of 2.1 mm or less and 100 mm in length for example) and particle sizes (usually less than 2 μm). Also, UHPLC employs smaller flow rates than HPLC, for example 0.2-0.7 ml/min. The smaller particles and reduced column diameter manifests itself in to higher backpressures in UHPLC compared to HPLC. HPLC instruments typically operate at maximum pressures of 400-600 bar, whilst UHPLC instruments can operate at up to 1500 bar. In principal, all of the above mentioned different types of liquid chromatography can also be conducted as UHPLC.

The stationary phase in chromatography is the phase over which the mobile phase passes. Stationary phases can be monolithic or particulate. A monolithic stationary phase is a continuous unitary porous structure prepared by in situ polymerization or consolidation inside the column tubing. Examples of monolithic phases include silica-based monoliths and organic polymer-based monoliths. In contrast, a particulate stationary phase consists of particles. The particles can also be silica-based or organic polymer-based. For both types of stationary phases the backbone silica or polymer can optionally be derivatized with functional groups. Thus, the surface is functionalized to convert it into a sorbent with the desired chromatographic binding properties. In the present case, the functionalization (which is synonymous to derivatization) may provide for better retention of specifically nucleic acids. A particularly preferred stationary phase material in monolithic or particulate form is poly(styrene-divinylbenzene). The poly(styrene-divinylbenzene) matric can be derivatized.

### DETAILED DESCRIPTION OF THE FINDINGS UNDERLYING THE PRESENT INVENTION

In order to ensure efficacy and safety of nucleic acid-based therapeutics (in particular RNA-based therapeutics), it is important to reliably and effectively measure the quality of the nucleic acid or nucleic acid mixture, which is to be used as a medicament. Such a quality control may be based on analyzing different aspects of the nucleic acid in the nucleic acid mixture such as the nucleic acid presence, nucleic acid integrity, nucleic acid quantity etc. In mixtures of therapeutic nucleic acids, the nucleic acid integrity is an especially important parameter as nucleic acids can degrade caused by e.g. heat, ribonucleases, pH or other factors, which reduces the integrity, leading to an impaired functionality of the nucleic acid.

The inventors of the present application found that the integrity of a nucleic acid mixture can be readily assessed by specifically analysing the nucleic acid with the largest size in that mixture, which e.g. corresponds to the “last peak” in a chromatographic analysis of the nucleic acid mixture. More specifically, the inventors found that the integrity of the nucleic acid with the largest size shows the fastest degradation amongst all nucleic acids in the nucleic acid mixture, meaning that the integrity of the nucleic acid with the largest size has a linear correlation with the integrity of the nucleic acid mixture such that the analysis of the nucleic acid with the largest size provides the result not only for the nucleic acid with the largest size but for the nucleic acid mixture as such. In other words, analysis of the nucleic acid with the largest size in a nucleic acid mixture provides information on the level of integrity of the nucleic acid most prone to degradation, i.e. information which shows the “worst” integrity in the overall mixture. It is exactly this “worst” integrity that provides the most meaningful information if a nucleic acid mixture should be used as a medicament. It is noted that nucleic acids in the mixture with higher integrities (i.e. with a lower degradation rate) are not relevant in the context of assessing the quality of a nucleic acid mixture.

In order to determine the integrity of the nucleic acid with the largest size, the amounts of the nucleic acid with the largest size determined prior to (and subjected to) the separation by size is compared to the actual amount of the nucleic acid with the largest size determined after the separation by size. Thus, for the determination of the integrity of the nucleic acid with the largest size, a “reference amount” is known, namely the amount as determined at an earlier time point to carried out prior to the present method (“t₀”). t₀ may be during production, after production or as part of a batch release control of the nucleic acid with the largest size and/or the nucleic acid mixture. The second time point (“t₁”) is the time point after separation by size. Between the two different time points t₀ and t₁, the integrity of the nucleic acid may have suffered, e.g. due to degradation. The extent of “suffering” can be assessed in the form of % integrity by comparing the amounts of the nucleic acids with the largest size at t₀ and t₁. It is the integrity or the % integrity of the nucleic acid with the largest size that is then also the integrity or % integrity of the nucleic acid mixture because of the correlation as outlined above.

A prerequisite for proper analysis of the integrity of a nucleic acid mixture (and the “last peak” which corresponds to the largest nucleic acid in said mixture in a chromatogram) is that the chromatographic or electrophoretic method separates the different nucleic acids in the nucleic acid mixture by size. Thus, any chromatographic or electrophoretic method is suitable for assessing the integrity of the nucleic acid with the largest size, which separates the at least two different nucleic acids present in the mixture by size (it is noted that, depending on the method, it must not necessarily be the “last peak”). The size difference is in particular due to a different number of nucleotides in the different nucleic acids in the nucleic acid mixture, but it may also be due to a different nucleotide composition that ultimately also results in different sizes.

The skilled person can choose from a variety of different chromatographic or electrophoretic methods. Chromatographic and electrophoretic methods are well-known in the art. In case chromatography is used, the analysis of the integrity of the nucleic acid with the largest size is based on determining the peak area (or “area under the peak”) of the corresponding chromatogram, in order to then determine the amount at t₁. Peak area can be determined by any suitable software which evaluates the signals of the detector system. The process of determining the peak area is also referred to as integration. Integration can be done automatically or manually using a suitable software. The peak area can e.g. be set in relation to the peak area of known amounts of the nucleic acid with the largest size in order to determine the amount. After comparing the gained integrity at t₁ to the integrity at t₀, the integrity at t₁ can be expressed in % integrity which corresponds to the difference in the integrity of the nucleic acid with the largest size at t₀ to the integrity at t₁.

If at least one further component is present in the nucleic acid mixture (for example if the nucleic acid mixture is comprised in a pharmaceutical formulation and/or encapsulated in lipid nanoparticles), the at least one further component may first need to be separated from the nucleic acid mixture prior to carrying out the method for determining the integrity of the nucleic acid mixture according to the invention. This can be done by any known separation technique in the art, i.e. precipitation by salt, chromatography, electrophoresis, etc.

### EXAMPLES

The following Examples are merely illustrative and shall describe the present invention in a further way. These Examples shall not be construed to limit the present invention thereto.

### Example 1: Preparation of RNAs

DNA sequences encoding five different target proteins with different sizes were introduced into a modified pUC19-derived vector backbone to comprise a 5′-UTR derived from the 32L4 ribosomal protein (32L4 TOP 5′-UTR) at the 5′-terminal end and a 3′-UTR derived from albumin, a histone-stem-loop structure, a stretch of adenine nucleotides (A64), and a stretch of cytosine nucleotides (C30) at the 3′-terminal end. Details of the five RNA sequences resulting from the transcription of the five different DNA sequences are provided in Table 1.

TABLE 1
Constructs used in the experiment

| Size of the RNA | | |
|---|---|---|
| (nucleotides) | G/C content | RNA ID |
| 667 | 62.22 | R3 |
| 925 | 62.49 | R1 |
| 1003 | 59.22 | R4 |
| 1621 | 63.50 | R2 |
| 1849 | 61.40 | R5 |

The five different DNA plasmids were linearized using EcoRI and transcribed in vitro using DNA dependent T7 RNA polymerase in the presence of a nucleotide mixture and cap analog under suitable buffer conditions. The final RNAs were obtained using an RNA manufacturing protocol implementing various quality controls on RNA and DNA level, essentially following the procedure as described in WO2016/180430.

The obtained five different RNAs were either analyzed individually (see example 3) or R1 and R2 were mixed in a mass ratio of 1:1 (R1:R2=1:1) to obtain an RNA mixture, which was also analyzed (see examples 4 and 5). The aim of the analysis was to determine the RNA integrity in a sample.

RNA mixtures may alternatively be produced as disclosed in WO2017/109134. In short, a mixture of different DNA constructs is used as a template for simultaneous RNA in vitro transcription to generate a mixture of different RNA constructs. Such an RNA mixture can also be subject to a further analysis as described herein when it comes to the integrity of the RNA mixture.

### Example 2: Determination of the RNA Integrity

Analytical HPLC was used to determine the integrity of the RNA and RNA mixture, respectively. For this analysis, RNA samples (i.e. a preparation comprising only a single RNA species or a preparation comprising an RNA mixture) were diluted to a concentration of 0.1 g/L using water for injection (WFI). 10 μl of the diluted RNA sample were injected into the HPLC column (monolithic poly(styrene-divinylbenzene) matrix).

The reversed-phase (RP) HPLC analysis was performed using the following conditions:

Gradient 1: Buffer A (0.1 M TEAA (pH 7.0)); Buffer B (0.1 M TEAA (pH 7.0) containing 25% acetonitrile). Starting at 30% buffer B the gradient extended to 32% buffer B in 2 min, followed by an extension to 55% buffer B over 15 minutes at a flow rate of 1 ml/min (adapted from WO2008/077592). Chromatograms were recorded at a wavelength of 260 nm.

The obtained chromatograms were evaluated using Chromeleon software and the relative peak area was determined in percent (%) as commonly known in the art (e.g., shape shoulder function; representative Example see FIG. 3). The relative peak area indicates the amount of integer RNA. Since the amount of the RNA injected into the HPLC is known (e.g. 100 ng), the analysis of the relative peak area provides information on the integrity of the RNA. Thus, if e.g. 100 ng are determined as the relative peak area, the integrity is 100%. If, however, the relative peak area corresponds to 80 ng, it is evident that the RNA has an integrity of 80%, mainly due to degradation taking place resulting in smaller fragments that are no longer encompassed by the respective peak area that is analyzed. The equipment used in the analytical HPLC is provided in Table 2.

TABLE 2
Materials used for analytical HPLC

| | |
|---|---|
| U3000 UHPLC-System | Thermo Scientific |
| HPLC column (monolithic poly(styrene- | Thermo Scientific |
| divinylbenzen) matrix | |
| WFI | Fresenius Kabi, Ampuwa |
| Acetonitril (MS-grade) | Fisher Scientific |
| 0.1M TEAA in WFI (Eluent A) | CureVac AG |
| 25% ACN in 0.1M TEAA (Eluent B) | CureVac AG |

### Example 3: The Integrity of RNA Species Differing in their Length after Degradation

Each of the purified RNAs R1-R5 (see example 1 and Table 1) was diluted to a concentration of 25 ng/ul in water for injection (WFI) and each RNA sample (comprising only a single RNA species) was heated to 85° C. to promote RNA degradation. After 10 minutes, the degraded RNA samples were individually analyzed via analytical HPLC as described in example 2.

Since a defined amount of RNA of each sample (namely 100 ng) was analyzed according to the conditions described in example 2, it is possible to determine the integrity of each sample. The integrity is reflected by the ratio of the amount of loaded RNA (i.e. 100 ng) to the amount of the recovered RNA (i.e. the relative area of the peak).

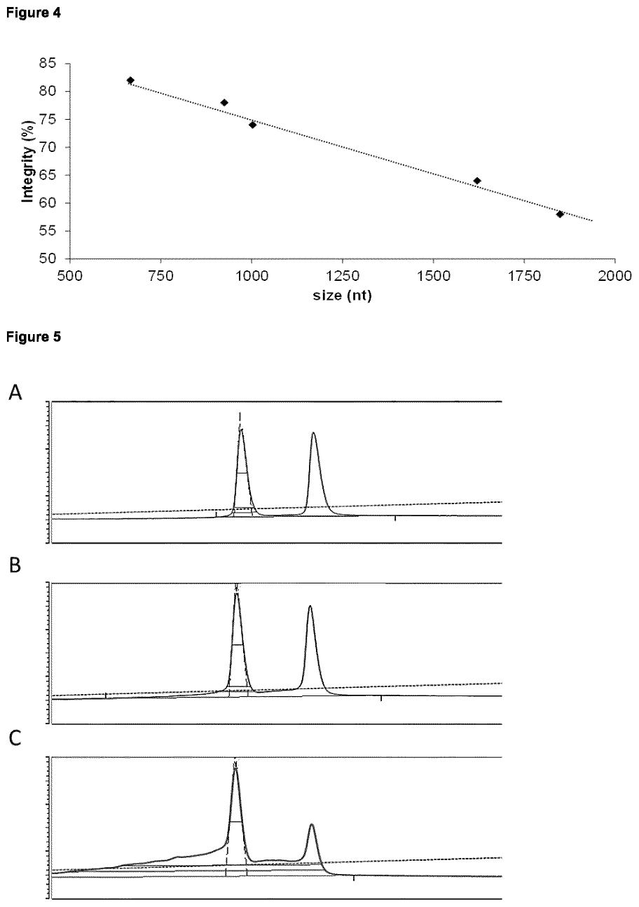

The results are shown in FIG. 4. When looking at the RNA with the smallest size, i.e. R3 with 667 nucleotides, an integrity of about 82% was determined, while an integrity of about 60% was determined for the RNA sample with the largest size, i.e. R5. In other words, the sample with the largest size showed a lower integrity meaning that it was degraded faster than the RNA of the smallest size. When taking all samples into account, it was found that the RNA size and the RNA integrity (in %) show a linear correlation (see FIG. 4).

Based on this finding, the inventors envisaged that it may be sufficient to analyze only the last peak of a chromatogram of an RNA mixture since the last peak represents the largest RNA species, which shows the fasted degradation kinetics. Accordingly, such an analysis provides information on the level of integrity of the RNA most prone to degradation, i.e. information that indicates the “worst” integrity rate in the overall sample. RNAs with higher integrity levels are not relevant here since it is the integrity of the overall mixture that is of interest. This is of course indicated by the lowest integrity value in the mixture. To test that model, RNA mixtures were generated, degraded, and analyzed via analytical HPLC (see Example 4).

### Example 4: Evaluation of the Last Peak Analysis for Integrity Determination of RNA Mixtures

Purified RNA constructs R1 and R2 were mixed in WFI in a ratio of 1:1 to generate an RNA mixture (final concentration: 50 ng/ul).

The RNA mixture was heated to 85° C. to degrade both RNA species comprised in the mixture. After 1 min (A), 10 min (B), and 40 min (C) time of degradation, the degraded RNA mixture was subjected to analytical HPLC as outlined in example 2. The obtained HPLC chromatograms are shown in FIGS. 5A, B, and C, illustrating the course of degradation of the RNA mixture over time (1 min=A; 10 min=B; 40 min=C).

It can be derived from FIG. 5 that, during the course of degradation, the relative peak area of the last peak (representing R2 with the larger size) in the chromatogram decreases, while the first peak in the chromatogram (representing R1) does not decrease but behaves differently.

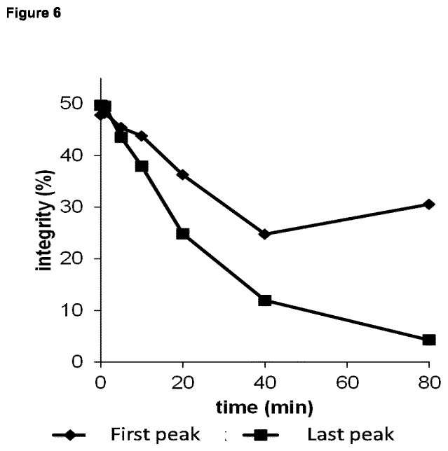

For each chromatogram, the integrity of both RNA species R1 and R2 comprised in the mixture was determined and plotted over time (shown in FIG. 6). The results in FIG. 6 show that the integrity of the last peak RNA (representing R2) follows the course of degradation, while the integrity of the first peak RNA (representing R1) does not follow the course of degradation.

Summarizing the above, the present Example shows that the determination of the integrity of the last peak of an RNA mixture on HPLC is sufficient to assess the integrity of the RNA mixture as a whole.

### Example 5: Confirmation of the Last Peak Analysis as Suitable Method for Determining the Integrity of RNA Mixtures

Purified RNA constructs R1 and R2 were mixed in WFI in a ratio of 1:1 to generate an RNA mixture (final concentration: 50 ng/ul). In addition, R1 and R2 were provided in separate preparations (final concentration of each preparation: 50 ng/ul).

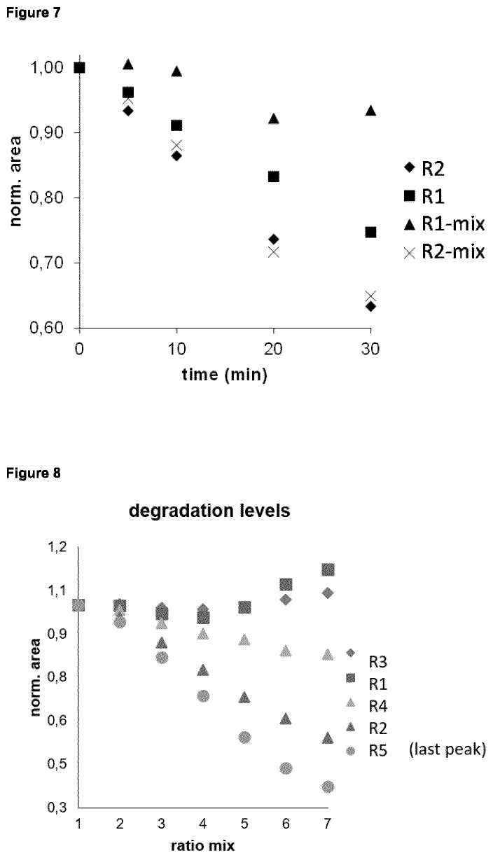

The RNA mixture was heated to 85° C. to degrade both RNA species comprised in the mixture. In addition, the two individual preparations comprising either R1 or R2 were heated to 85° C. to degrade the RNA species in each preparation. Samples were prepared at different time points in order to reflect the course of degradation over time (5 min, 10 min, 20 min and 30 min, see FIG. 7). Degraded samples (RNA mixture, individual RNA species) were analyzed on HPLC at the different time points as outlined in example 2 and the obtained values were plotted over degradation time as shown in FIG. 7.

FIG. 7 shows the peak area (normalized over all three samples) of RNA R2 and RNA R1, determined either in the individual samples (R1, R2) or in the mixture (R1-mix, R2-mix). The peak area is again indicative for the integrity of the RNA in the respective sample, i.e. a normalized peak area of 1 means that the RNA in the sample has 100% integrity.

It can be derived from FIG. 7 that the normalized peak areas for the R2 RNA species, the RNA with the larger size, show an almost identical course of degradation when determined in the RNA mixture (comprising R1 and R2, see R2-mix) as well as when measured in the individual R2 RNA sample (R2). When it comes to R1, the RNA with the smaller size, the normalized peak area for the R1 RNA species determined for the individual R1 RNA sample (R1) shows an expected course of degradation, while the values obtained from the RNA mixture (comprising R1 and R2, see R1-mix) strongly deviate from the expected values.

Summarizing the above, the present Example demonstrates that determining the integrity of the last peak of an RNA mixture on HPLC is sufficient to determine the integrity of the RNA mixture as a whole.

### Example 6: Last-Peak Analysis (LPA) Using Formulated RNA Mixtures

### 6.1 LPA of an LNP-Encapsulated RNA Mixture

A lipid nanoparticle (LNP)-encapsulated RNA mixture is prepared using an ionizable amino lipid (cationic lipid), phospholipid, cholesterol and a PEGylated lipid. LNPs are prepared as follows: Cationic lipid, DSPC, cholesterol and PEG-lipid are solubilized in ethanol. Briefly, the RNA mixture obtained according to Example 1 (i.e. a mixture of R1 and R2 or a mixture comprising all five RNAs, R1 to R5) is diluted to a total concentration of 0.05 mg/ml in 50 mM citrate buffer, pH4. Syringe pumps are used to mix the ethanolic lipid solution with the RNA mixture at a ratio of about 1:6 to 1:2 (vol/vol). The ethanol is then removed and the external buffer is replaced with PBS by dialysis. Finally, the lipid nanoparticles are filtered through a 0.2 μm pore sterile filter. Lipid nanoparticle particle diameter size is determined by quasi-elastic light scattering using a Malvern Zetasizer Nano (Malvern, UK).

A sample of an LNP-formulated RNA mixture is treated with a detergent (about 2% Triton X100) to dissociate the LNPs. The released RNA mixture is captured using Agencourt AMPure XP beads (Beckman Coulter, Brea, CA, USA) essentially according to the manufacturer's instructions. Following preparation of the RNA mixture, LPA is performed (as described in previous Examples) to determine the integrity of said RNA mixture.

### 6.2 LPA of a Protein-Polymer-Complexed RNA Mixture

20 mg peptide (CHHHHHHRRRRHHHHHHC-NH2, SEQ ID NO: 45 of published PCT application WO2018211038) as TFA salt is dissolved in 2 ml borate buffer pH8.5 and stirred at room temperature (RT) for approximately 18 h. Then, 12.6 mg PEG-SH 5000 (Sunbright) dissolved in N-methylpyrrolidone is added to the peptide solution and filled up to 3 ml with borate buffer pH8.5. After 18 h incubation at RT, the mixture is purified and concentrated by centricon procedure (MWCO 10 kDa), washed against water, and lyophilized. The lyophilisate is dissolved in ELGA water and the concentration is adjusted to 10 mg/ml. The obtained polyethylenglycol/peptide polymers (HO-PEG5000-S-(S-CHHHHHHRRRRHHHHHHC-S-)7-S-PEG5000-OH) are then formulated with the RNA mixture as obtained in Example 1 (i.e. a mixture of R1 and R2 or a mixture comprising all five RNAs, R1 to R5).

First, ringer lactate buffer and respective amounts of the obtained polymer are mixed to generate polymer carriers. Then, the final mRNA carrier system is assembled by mixing the RNA mixture with respective amounts of polymer-lipid carrier. After 10 min incubation at room temperature, polymer-complexed RNA particles are formed.

After high salt treatment and/or heparin treatment of nanoparticles, complexed RNA is released. The released RNA mixture is captured using Agencourt AMPure XP beads (Beckman Coulter, Brea, CA, USA) essentially according to the manufacturer's instructions. Following preparation of the RNA mixture, LPA is performed (as described in previous Examples) to determine the integrity of said RNA mixture.

### Example 7: Analysis of the Integrity of an RNA Mixture Comprising 5 Different RNAs Using the Last Peak Analysis

The five different RNAs of example 1, R1 to R5, were mixed in order to arrive at an RNA mixture comprising five different RNAs and the integrity of the mixture at different stages of degradation was then investigated as described in the following.

Thus, a 5er RNA mixture comprising R1 to R5 of example 1 was generated in line with Example 1 (total RNA concentration 0.2 μg/μl, 320 μl total volume). 160 μl of said mixture was stored on ice (in order to prevent any degradation), and 160 μl of said mixture was heated at 85° C. for 40 minutes (in order to promote degradation). Mixtures reflecting different stages of degradation were then generated by mixing (i) a sample of the non-degraded RNA mixture (i.e. the ice sample) with (ii) a sample of degraded RNA mixture (i.e. the heated sample), wherein the ratios of the two samples ranged from 10:0 to 0:10. The obtained RNA mixtures reflecting different degradation stages 1 to 7 are shown in Table 3.

TABLE 3
Amounts of RNA derived from the ice sample and
the 85° C. sample, resulting in different mixes

| | Mix | Ice sample [mg] | 85° C. sample [mg] |
|---|---|---|---|
| 1 | 10:0 | 40 | 0 |
| 2 | 9:1 | 44.1 | 5.0 |
| 3 | 7:3 | 34.8 | 14.6 |
| 4 | 5:5 | 25.3 | 25.0 |
| 5 | 3:7 | 15.1 | 34.5 |
| 6 | 1:9 | 4.8 | 44.2 |
| 7 | 0:10 | 0 | 40 |

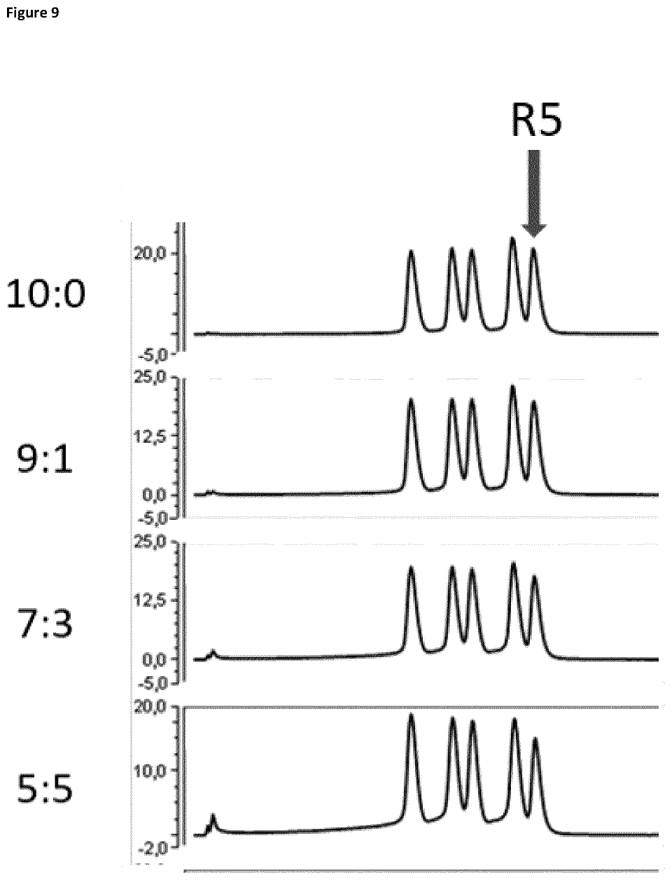

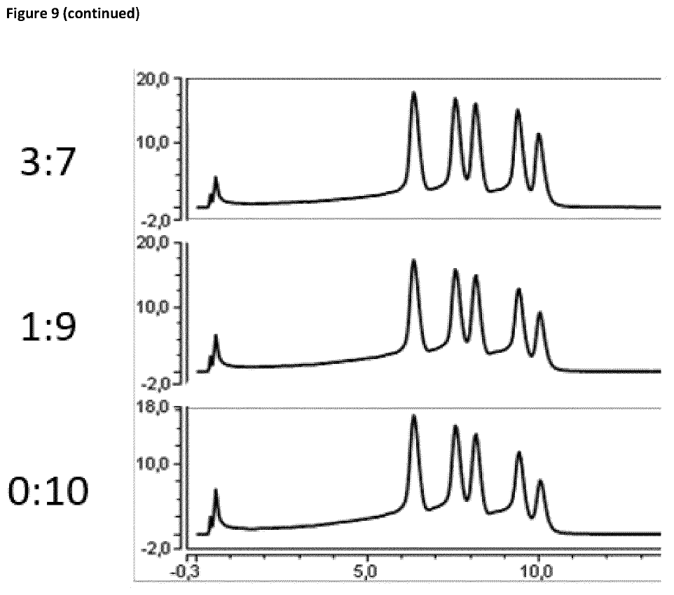

RNA mixtures 1 to 7 according to Table 3 were analyzed on HPLC as outlined in example 2. The obtained HPLC chromatograms for each mixture are provided in FIG. 9, wherein the peak of the RNA with the largest size, R5, is indicated by an arrow. It is immediately evident that the peaks drastically differ between the different preparations. The respective peak areas of each individual RNA component were determined and plotted for each RNA of each RNA mixture (see FIG. 8).

As shown in FIGS. 8 and 9, the integrity of the last peak in the HPLC chromatogram (RNA R5) decreases with decreasing integrity of the mixture. Accordingly, the integrity of R5 correlates with mixture integrity. Notably, the other RNAs do not correlate with the reduced integrity of the mixture. Thus, e.g. R1 and R3 even show an increase in their peak areas by about 10-15%, as can be derived in particular from FIG. 8.

Accordingly, the integrity of the last peak in the HPLC chromatogram (the peak corresponding to R5, the RNA with the largest size) can be used to reliably determine integrity of the mixture as a whole.

1. A method for determining the integrity of a nucleic acid mixture comprising at least two nucleic acids with different sizes, the method comprising the following steps:
a) subjecting the nucleic acid mixture comprising at least two nucleic acids with different sizes to a method for separation by size;
b) determining the integrity of the nucleic acid with the largest size; and
c) determining the integrity of the nucleic acid mixture by assigning the integrity of the nucleic acid with the largest size determined in step b) to the nucleic acid mixture.

2. The method according to claim 1, wherein the method for separation by size is a chromatographic method or electrophoresis.

3. The method according to claim 2, wherein the chromatographic method is liquid chromatography selected from the group consisting of normal phase liquid chromatography, reversed phase liquid chromatography (RPLC), ion-exchange liquid chromatography, ion-pair reversed-phase liquid chromatography (IP-RPLC), size-exclusion chromatography (SEC), high-performance liquid chromatography (HPLC), reversed phase high-performance liquid chromatography (RP-HPLC), ion-pair reversed-phase high-performance liquid chromatography (IP-RP-HPLC), ultra-high performance liquid chromatography (UHPLC), hydrophilic interaction chromatography (HIC), hydrophilic interaction liquid chromatography (HILIC), and combinations thereof.

4. The method according to claim 2, wherein the liquid chromatography is high-performance liquid chromatography (HPLC), reversed phase high-performance liquid chromatography (RP-HPLC) or ion-pair reversed-phase high-performance liquid chromatography (IP-RP-HPLC).

5. The method according to claim 4, wherein a monolithic stationary phase or a particulate stationary phase is used in the liquid chromatography.

6. The method according to claim 5, wherein the monolithic stationary phase or the particulate stationary phase comprises a poly(styrene-divinylbenzene) matrix.

7. The method according to claim 4, wherein the at least two nucleic acids with different sizes differ by at least 20 nucleotides.

8. The method according to claim 4, wherein the integrity of the nucleic acid with the largest size is determined by comparing the amount of the nucleic acid with the largest size determined prior to the separation by size to the actual amount of the nucleic acid with the largest size determined after the separation by size.

9. The method according to claim 8, wherein the amount of the nucleic acid with the largest size determined prior to the separation by size is known from the production of the nucleic acid with the largest size and/or the production of the nucleic acid mixture comprising the nucleic acid with the largest size.

10. The method according to claim 8, wherein the actual amount of the nucleic acid with the largest size is determined by analysing the fraction of the nucleic acid with the largest size obtained by a chromatographic or electrophoretic method.

11. The method according to claim 10, wherein the analysing comprises determining the area under the peak of the fraction of the nucleic acid with the largest size obtained by a chromatographic method.

12. The method according to claim 11, wherein the area under the peak of the fraction of the nucleic acid with the largest size is integrated.

13. The method according to claim 12, wherein the integrated peak of the fraction of the nucleic acid with the largest size indicates the actual amount of the nucleic acid with the largest size.

14. The method according to claim 4, wherein the integrity is indicated in % integrity.

15. The method according to claim 4, wherein the nucleic acid mixture comprising at least two nucleic acids with different sizes is comprised in a formulation comprising at least one further component, and wherein the nucleic acid mixture is separated from the at least one further component prior to step a).

16. The method according to claim 15, wherein the at least one further component is selected from the group consisting of a lipid, a protein, a peptide, a cationic compound, a polycationic compound, and combinations thereof.

17. The method according to claim 16, wherein the at least one further component is a lipid nanoparticle or a cationic or polycationic peptide or a cationic or polycationic protein.

18. The method according to claim 17, wherein the nucleic acid is RNA.

19-24. (canceled)

25. The method according to claim 6, wherein the nucleic acid is RNA.

26. The method according to claim 25, wherein the nucleic acid mixture comprising at least two nucleic acids with different sizes is comprised in a formulation comprising lipid nanoparticles, and wherein the nucleic acid mixture is separated from the lipid nanoparticles prior to step a).
